(19) United States
(12) Patent Application Publication
(10) Pub. No.: US 2008/0262525 A1
(43) Pub. Date: Oct. 23, 2008

(54) TISSUE PENETRATION AND GRASPING APPARATUS

(75) Inventors: Arvin T. Chang, West Covina, CA (US); Richard C. Ewers, Fullerton, CA (US)

Correspondence Address: LEVINE BAGADE HAN LLP, 2483 EAST BAYSHORE ROAD, SUITE 100, PALO ALTO, CA, 94303, US

(73) Assignee: USGI Medical, Inc., San Clemente, CA (US)

(21) Appl. No.: 11/736,541

(22) Filed: Apr. 17, 2007

(51) Int. Cl. A61B 17/32

(52) U.S. Cl. 606/170

(57) ABSTRACT

A tissue grasping apparatus includes a control member, an elongated shaft, and a tissue penetrating and grasping member attached to the distal end of the elongated shaft. An activation mechanism provides an user-operable connection between the control member and the tissue penetrating and grasping member. In an embodiment, the tissue penetrating and grasping member includes a rigid penetrating member that is rotatably attached to the distal end of the elongated shaft. In an embodiment, the activation mechanism includes a flexible drive wire attached to the penetrating member.

### RELATED APPLICATION DATA

None.

### FIELD OF THE INVENTION

The present invention relates to surgical instruments used to engage, penetrate, grasp, or manipulate tissue, and methods of their use.

### BACKGROUND OF THE INVENTION

Tissue engaging, grasping, and manipulating instruments are used during open surgery, laparoscopic surgery, endoscopic surgery, or translumenal surgery. A common type of instrument available for endolumenal acquisition of stomach tissue is an endoscopic grasper. A typical endoscopic grasper includes a pair of hinged jaws located at the distal end of a flexible shaft. The jaws are actuated between open and closed positions. Typically, the jaws are actuated using a push/pull rod or wire that extends through the flexible shaft to connect to the jaws via a mechanical linkage. When the jaws are opened, they assume a wide “V” shape. The jaws are then brought into contact with tissue, after which the jaws are actuated to the closed position. Closing the jaws causes the jaws to catch on, pinch, or entrap the tissue.

Conventional hinged jaw-type endoscopic graspers like those described above have several limitations. For example, the mechanical linkages used to actuate the jaws in typical endoscopic graspers are unable to drive the jaws open to or beyond an included angle (the angle formed between the jaws) of 180 degrees. This limitation reduces the effectiveness of these graspers in circumstances in which a wider throw (having an included angle equal to or greater than 180 degrees) is desirable. In addition, the mechanical linkages must be configured such that they do not reach a point of linear alignment during actuation to the closed position, otherwise the closure force will drop to zero and the jaws will be inoperable.

Another common type of endoscopic grasper includes two or more spring biased jaws that are actuated using an external sleeve. The jaws comprise flats of spring steel that have opposing curved or angled surfaces that have a spring bias toward the open position relative to one another. The external sleeve is slidable over the jaws. As the external sleeve is translated distally toward the ends of the spring biased jaws, the external sleeve causes the jaws to move toward one another to the closed position.

The foregoing spring jaw-type of endoscopic grasper also has limitations. For example, the jaws of these types of graspers open passively, i.e., they open only due to and are only as strong as the inherent spring force between the jaws. They are, therefore, not well suited to open fully in constrained spaces where surrounding tissue could retard the spring open force. Also, the closure requires a relative motion that makes targeting of a selected portion of tissue (or other target) difficult due to the relative movement (e.g., retraction) of the jaws into the external sleeve. Further still, the closure force of the jaws reaches its peak as the jaws are being retracted fully into the external sleeve, at which point the jaws are unable to grasp tissue.

Yet another type of endoscopic grasper includes a tissue piercing coil member attached to the distal end of a flexible shaft. The coil member has a sharp tip and an open pitch that allows the coil member to penetrate tissue when it is rotated against the tissue with a light amount of distal force. Once tissue is penetrated, the grasper allows the user to manipulate the tissue by advancing or retracting the grasper.

The coil-type grasper has limitations in that it only grasps a single point of tissue, and cannot easily grasp or bring together multiple contact points or grasp a relatively large area of tissue. The coil-type grasper also achieves its grasp by a “blind” penetration of tissue by the coil.

### SUMMARY

In one general aspect, a medical instrument according to the present invention includes a tissue engaging, penetrating, grasping, and manipulating member configured for introduction into a patient. The medical instrument is adapted for use during open surgery, laparoscopic surgery, endoscopic surgery, or translumenal surgery. In several preferred embodiments, the medical instrument has a small profile such that the tissue grasping member is able to pass through a small diameter lumen to be routed to a site within a patient's body. In several other preferred embodiments, the medical instrument has an elongated, flexible shaft that allows the instrument to be passed through tortuous anatomy, either as a standalone instrument or as an instrument to be passed through a lumen of an overtube. The tissue grasping member is used to engage, penetrate, grasp, acquire, position, or otherwise manipulate tissue within a patient. The medical instrument is suitable for use as a standalone instrument, or it may be used in combination with other instruments that provide independent or related functions.

In several embodiments, the medical instrument includes a tissue penetrating member rotatably attached to the distal end of an elongated, flexible shaft. An activation mechanism is operatively coupled to the tissue penetrating member, and is also responsive to a control member, such as a handle. The user is able to manipulate the handle to operate the tissue penetrating member.

In an embodiment, the tissue penetrating member comprises a rigid needle. The needle includes a body member and a sharp, penetrating tip portion. In several embodiments, the tip portion includes a conical shape, a pyramidal shape, or a faceted, beveled needle tip formed of stainless steel having a caliber of 18 gauge or smaller. The tissue penetrating member is attached either directly or indirectly to a distal portion of the elongated, flexible shaft such that the tissue penetrating member is able to rotate through an engagement angle relative to the longitudinal axis of the shaft. In an embodiment, the tissue penetrating member is configured to slide longitudinally within a slot formed at or near the distal end of the shaft.

In a second general aspect, a method for engaging, penetrating, grasping, and/or manipulating tissue includes the steps of providing a medical instrument having a tissue penetrating member at a location adjacent to a tissue site, moving the medical instrument to cause the tissue penetrating member to penetrate the tissue, rotating the tissue penetrating member through an engagement angle, and manipulating the medical instrument in order to push, pull, or otherwise move the tissue from its natural position. In several embodiments, the method is performed using a medical instrument that is placed near the tissue site either endoscopically, laparoscopically, or during open surgery. In an embodiment, the medical instrument is advanced to a tissue site via a natural body orifice.

### BRIEF DESCRIPTION OF THE DRAWINGS

FIG. 1A is a perspective view of a medical instrument having a tissue penetrating and grasping member in accordance with the present invention.

FIGS. 1B and 1C are cross-sectional views of two embodiments of a shaft in accordance with the medical instrument shown in FIG. 1A.

FIG. 1D is a cross-sectional view of a handle suitable for use with the medical instrument shown in FIG. 1A.

FIGS. 2A-C are side views of an embodiment of a medical instrument having a tissue penetrating and grasping member.

FIGS. 3A-C are side views of additional embodiments of medical instruments having a tissue penetrating and grasping member.

FIGS. 4A-B are side views of another embodiment of a medical instrument having a tissue penetrating and grasping member.

FIGS. 5A-C are side views of additional embodiments of medical instruments having a tissue penetrating and grasping member.

FIGS. 6A-B are side views of another embodiment of a medical instrument having a tissue penetrating and grasping member.

FIGS. 7A-B are side views of another embodiment of a medical instrument having a tissue penetrating and grasping member.

FIGS. 8A-B are side views of another embodiment of a medical instrument having a tissue penetrating and grasping member.

FIGS. 9A-D are side views of another embodiment of a medical instrument having a tissue penetrating and grasping member.

FIGS. 10A-C are side views of another embodiment of a medical instrument having a tissue penetrating and grasping member.

FIGS. 11 and 12 are side views of additional embodiments of medical instruments having a tissue penetrating and grasping member.

FIGS. 13A-B are side views of another embodiment of a medical instrument having a tissue penetrating and grasping member.

### DETAILED DESCRIPTION

The devices described herein include several embodiments of medical instruments that are adapted to engage, penetrate, grasp, and/or manipulate tissue. The medical instruments are adapted for use with soft tissue found in human or animals, or with other tissue such as cartilage, muscle, soft areas of bone, or others. In several embodiments, the medical instrument includes a penetrator adapted to penetrate tissue. After penetrating tissue to a desired depth, the penetrator is moved in a direction away from the direction of penetration through the tissue, such as through an arcuate or curvilinear path. The arcuate or curvilinear movement causes the penetrator to engage and/or grasp the tissue such that the tissue is able to be manipulated by the medical instrument under control of the user. After the desired manipulation is completed, the penetrator is returned to its original position so as to release the tissue.

In several embodiments, the medical instruments are configured to be able to pass through a relatively small diameter lumen such as the surgical tool lumens provided during laparoscopic, endoscopic, or translumenal surgery. In other embodiments, the instrument is configured for use during conventional open surgery, or other procedures in which the size restraints required during laparoscopic, endoscopic, or translumenal surgery are not present.

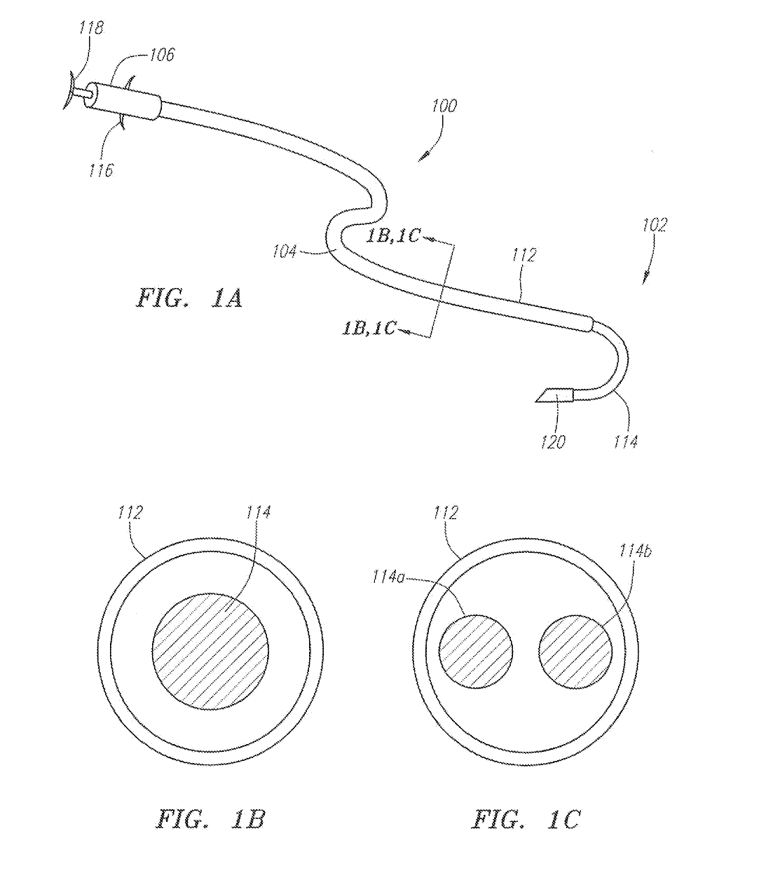

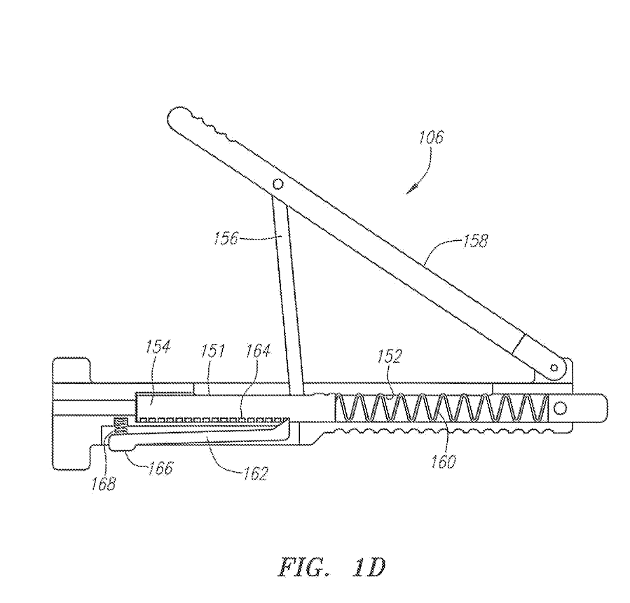

Referring to FIG. 1A, a first embodiment of a medical instrument 100 for engaging, penetrating, grasping, and/or manipulating tissue is shown. The medical instrument shown in FIG. 1A includes an end effector 102 attached to the distal end of a shaft 104. In the embodiment shown in FIG. 1A, the end effector 102 includes a tissue penetrating member and a deployment mechanism, each of which is described more fully herein. A control member, such as a handle 106, is provided at the proximal end of the instrument, preferably coupled to the proximal end of the shaft 104. The control member serves as an interface for the user to manipulate or control the action of the tissue grasping member 102.

In an embodiment, the shaft 104 is an elongated, flexible member having an external sleeve 112 and an internal pusher 114. (See FIGS. 1B and 1C). The sleeve 112 and pusher 114 are capable of longitudinal motion relative to one another. For example, in an embodiment, the sleeve 112 is cylindrical, defining an internal lumen in which the pusher 114 is located. The pusher is longitudinally translatable within the external sleeve, preferably slidably, thereby providing the capability for the external sleeve 112 and pusher 114 to move longitudinally relative to one another.

The external sleeve 112 is adapted to provide a flexible, operable interconnection between the handle 106 and the end effector 102. In an embodiment, the external sleeve 112 is formed of materials having sufficient strength and other materials properties to support transmission of torque forces between the handle 106 and the end effector 102. For example, the external sleeve 112 is capable of causing the end effector 102 to rotate around the longitudinal axis of the shaft 104 in response to a rotation of the handle 106. In an embodiment, the external sleeve 112 also supports relative sliding movement of the pusher 114 within the sleeve with very little friction and without a large amount of longitudinal stretch or contraction of the shaft 104. In an embodiment, the external sleeve 112 is constructed of a single material. In another embodiment, the external sleeve 112 has a composite construction that includes two or more of a main body material to provide structure and flexibility, a reinforcing material to provide torque transmission capability and/or to reduce or eliminate stretch and contraction, and a liner material to reduce friction and/or to reduce or eliminate stretch and contraction. Examples of materials that are suitable for forming the main body portion of the external sleeve include polymeric materials, such as polyester amide block copolymer (PEBAX™), nylon, polyurethane, or other similar materials commonly used for medical instrument applications. Examples of suitable reinforcing materials include polymeric or metallic braid materials and/or reinforcing wires. Examples of suitable liner materials include polytetrafluoroethylene (PTFE), polyetheretherketone (PEEK), or other suitable materials.

The pusher 114 is adapted to transfer a longitudinally-directed force applied by the user from the handle 106 to the tissue grasping member 102. In the embodiment shown in FIG. 1B, the pusher 114 is formed of a single solid wire, coiled wire, or similarly-shaped member that extends through the length of the lumen formed by the external sleeve 112. As described above, the pusher 114 and external sleeve 112 are adapted to move longitudinally relative to one another. In an embodiment, the pusher 114 is a wire formed of stainless steel, nickel titanium alloy (Nitinol), or other material commonly used for medical instrument applications. In other embodiments, the pusher 114 is formed of non-continuous segments aligned end-to-end and joined together to provide the desired longitudinal translation force. In still other embodiments, such as that shown in FIG. 1C, the pusher 114 comprises two or more continuous or non-continuous wires, rods, or similarly-shaped members 114a, 114b. In some embodiments, the two or more members are arranged coaxially within the sleeve 112, while in other embodiments the two or more members 114a, 114b are aligned alongside one another, as shown in FIG. 1C.

The handle 106 is configured to provide relative movement between the external sleeve 112 and the pusher 114 associated with the shaft 104. Several common types of medical instrument handles are suitable for this purpose. In FIG. 1A, the medical instrument 100 is illustrated with a syringe-type handle 106 having a main body 107 connected at its distal end to the proximal end of the external sleeve 112, and a pair of finger tabs 116 extending from opposite sides of the main body 107. A thumb tab 118 is attached to the pusher 114 and extends out of the proximal end of the handle main body 107. The syringe-type handle 106 is a common handle used in medical instruments that require relative movement between a pair of shafts or a sleeve and pusher, such as the present device. Other handle types are suitable for use as well, as will be recognized by a person having skill in the art. For example, in other embodiments, the handle includes either a pistol grip, a grip having tabs and a thumb plunger, or other structures. In still other embodiments, the handle includes a spring providing a biasing force between the sleeve 112 and the pusher 114, the spring causing the tissue grasping member 102 to be biased to an open or closed position. In still other embodiments, the handle 106 includes an indexing mechanism used to activate the end effector 102 to one or more predetermined positions. In still other embodiments, the handle 106 includes a locking mechanism to selectively lock the end effector 102 in a selected position.

Turning to FIG. 1D, another embodiment of a handle 106 is illustrated. The handle 106 includes an elongated main body 150 having a central channel 152 in which a pusher block 154 is slidably received. A link arm 156 extends through a slot 151 formed on the upper surface of the main body in communication with the central channel 152, and is pivotably attached at one end to the upper surface of the pusher block 154, and pivotably attached at its other end to an actuation arm 158. The actuation arm 158 is pivotably attached to the upper surface of the main body 150 near its distal end. A spring 160 is located within the central channel 152 near its distal end, and provides a spring force biasing the pusher block 154 proximally within the channel.

The main body 150 of the handle 106 is attached or otherwise connected to the external sleeve 112 of the shaft 104. The pusher block 154 is attached or otherwise connected to the pusher 114. Accordingly, as the pusher block 154 is advanced (distally) or withdrawn (proximally) within the central channel 152, the pusher 114 is advanced or withdrawn relative to the external sleeve 112. In the embodiment shown, a user applied downward force applied to the actuation arm 158 causes the pusher block 154 to advance (distally) against the force of the spring 160, thereby advancing the pusher 114 within the sleeve 112. When the user applied force on the actuation arm 158 is released, the spring 160 causes the pusher block 154 to withdraw, thereby withdrawing the pusher 114 relative to the sleeve 112. As explained herein, this motion creates the actuation forces controlling the operation of the tissue grasping member 102.

In the embodiment shown in FIG. 1D, a ratchet mechanism is provided on the handle 106 to selectively and releasably restrict the pusher block 154 to move in only a single direction within the main body 152. The ratchet mechanism includes a pawl 162 that is pivotably connected to the bottom surface of the main body 150 of the handle 106, and is adapted to selectively engage one of a plurality of slots 164 formed on the underside of the pusher block 154. A pawl spring 168 is located between the pawl 162 and the handle main body 152 and provides a force biasing the pawl 162 into engagement with the pusher block 154. When the pawl 162 is engaged with one of the slots 164, the pusher block 154 is unable to move proximally in the central channel 152. The user is able to disengage the pawl 162, thereby releasing the pusher block 154, by applying a force on the release end 166 of the pawl, which is exposed on the underside of the handle main body. The ratchet mechanism may be reversed—i.e., to restrict distal movement of the pusher block 154 by reversing the relative engagement of the pawl 162 with the slots 164 shown in FIG. 1D, as will be recognized by a person skilled in the art. The ratchet mechanism may be used to maintain a releasable opening or closing force on the tissue grasping member 102, as desired.

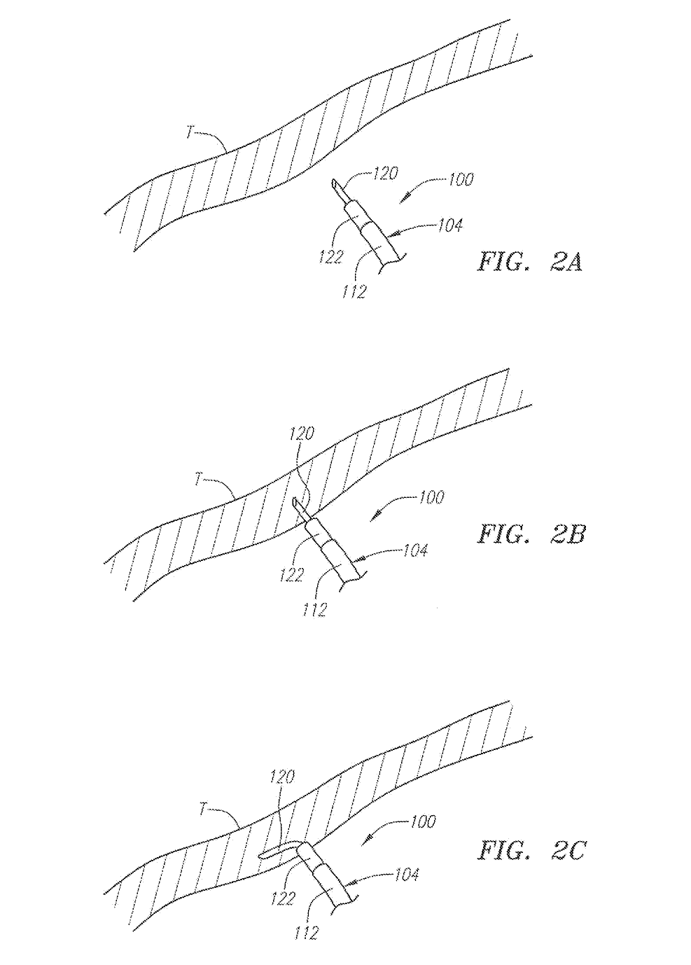

Turning to FIGS. 2A-C, an embodiment of an end effector 102 is shown. The end effector 102 is shown located at the distal end of the shaft 104, and includes a penetrator 120 at the distal end of the pusher 114 and a stop member 122 at the distal end of the sleeve 112. In the embodiment shown, the penetrator 120 is formed integrally with the pusher 114, although in other embodiments the penetrator is formed as a separate component that is connected or otherwise attached to the distal end of the pusher 114. The penetrator 120 includes a sharpened distal tip to facilitate penetration into tissue T. The penetrator 120 is preferably formed of a material having sufficient strength to penetrate tissue, such as a metal or polymer. Examples of suitable materials include stainless steel and nickel titanium alloy (Nitinol). In the embodiment shown, the stop member 122 is formed integrally with the sleeve 112, although in other embodiments the stop member 122 is formed as a separate component that is connected or otherwise attached to the distal end of the sleeve 112. The stop member 122 has a transverse dimension (e.g., diameter) that is larger than the transverse dimension of the penetrator 120 and is preferably provided with a blunt distal edge adapted to engage tissue without penetrating the tissue. In some embodiments, the stop member 122 is formed of a single material, such as the polymeric or metallic materials described herein. In other embodiments, the stop member 122 is a composite construction.

In an embodiment, the penetrator 120 is of a fixed length and position relative to the stop member 122. In another embodiment, the penetrator 120 is movable longitudinally under a force applied by the pusher 114 from a position in which it is enclosed by the stop member to a position in which it extends a distance distally of the stop member 122.

To operate the medical instrument 100, the end effector 102 is advanced until it is adjacent to a tissue site, as shown in FIG. 2A. If the end effector 102 includes a penetrator 120 that is capable of being moved inside and outside the sleeve 112, then the penetrator 120 is advanced distally to be exposed from within the sleeve 112 and the stop member 122. The medical instrument 100 is then advanced distally until the penetrator 120 penetrates the tissue T to a desired depth, as shown in FIG. 2B. The penetrator 120 is then activated to change direction relative to its initial penetration direction, as shown in FIG. 2C. This movement places the penetrator 120 at an angle relative to the tissue surface, effectively engaging, grasping, and/or trapping the tissue located above the penetrator 120. As a result, the medical instrument 100 can then be manipulated to push, pull, or torque the engaged tissue. By transforming the penetrator back to its initial position (as shown, for example, in FIG. 2B) or relatively similar to its initial position, the penetrator 120 is configured to be withdrawn from the tissue T.

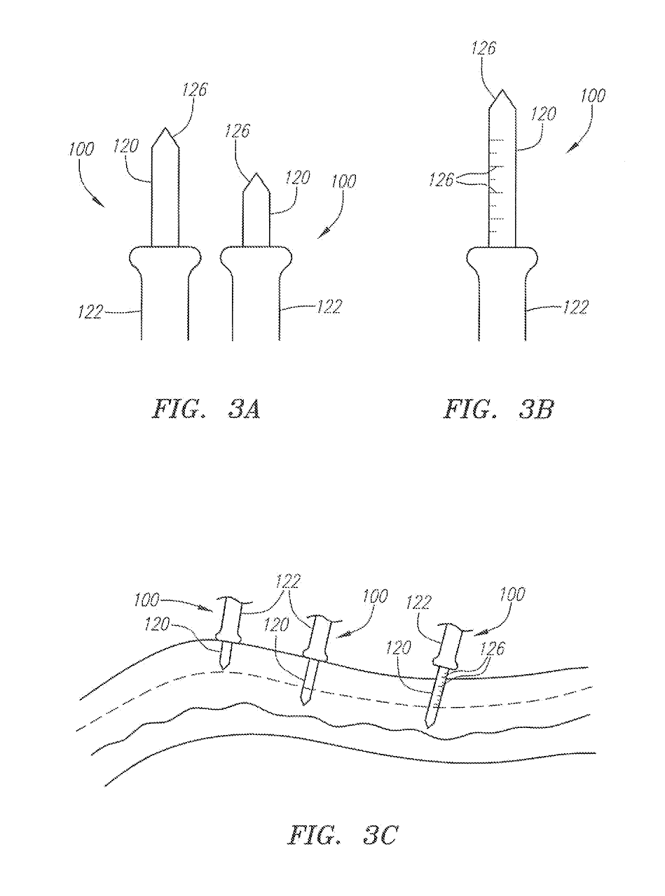

In some embodiments, the penetrator 120 has a fixed length. Accordingly, the user is able to select a medical instrument having a penetrator 120 with a length that is suitable for the clinical environment. A pair of medical instruments 100, each having a penetrator 120 of different length, are shown in FIG. 3A. In the embodiments shown, the penetrator 120 is of a desired length and the stop member 122 has a size and shape that prevents the stop member 122 from penetrating tissue. In this manner, the user is able to advance the instrument into the target tissue until the stop member 122 butts up against the tissue T, at which point the penetrator 120 will have reached its desired depth. In the embodiment shown in FIG. 3B, the penetrator 120 is provided with depth markings 124 that allow the user to visually determine the depth of penetration of the penetrator 120. The medical instruments 100 illustrated in FIGS. 3A and 3B are shown engaged in tissue T in FIG. 3C.

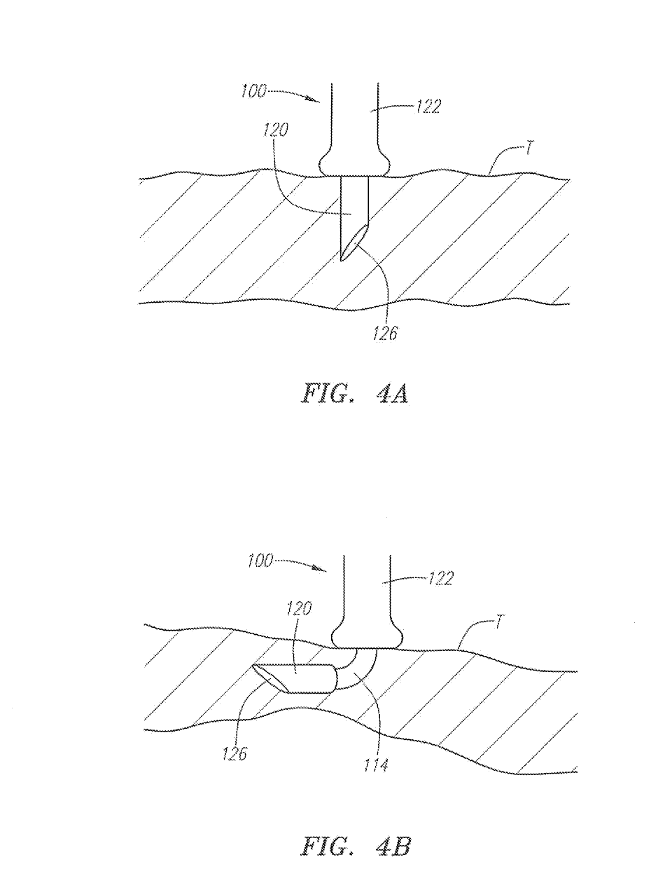

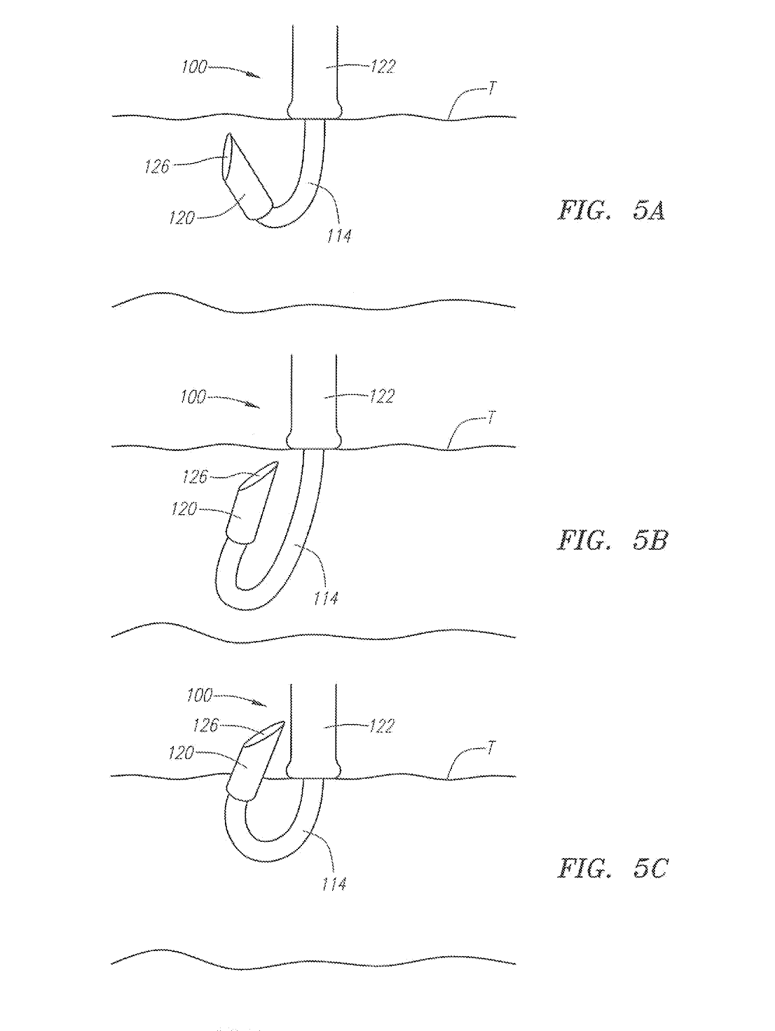

The penetrator 120 is constructed to penetrate tissue. In several embodiments, the penetrator 120 comprises a rod having a tissue penetrating tip, a wire having a tissue penetrating tip, or a ribbon having a tissue penetrating tip. In several embodiments, the penetrating tip 126 comprises a conical, pyramidic, beveled, or faceted needle or obturator type tip. In other embodiments, the penetrating tip 126 is blunt and is operably connected to an electrosurgical cutting current or an ultrasonic vibrator. In an embodiment, shown in FIGS. 4A-B, the penetrating tip 126 comprises a faceted, beveled needle tip formed of stainless steel having a caliber of 18 gauge or smaller. The bevel direction is such that it angles away from the inside curve of the direction that the penetrator 120 takes during engagement. (See FIG. 4B). This positions the bevel angle away from the direction the penetrator 120 would be pulled to manipulate tissue, thereby reducing the tendency for the tissue to initiate slipping because the holding tissue is faced with a straight surface. This direction for the bevel also facilitates return of the penetrator 120 to the straight axial position, shown in FIG. 4A.

After tissue penetration, the penetrator 120 advances through tissue at an engagement angle and engagement direction determined by the materials and construction of the end effector 102. In several embodiments, the engagement angle and engagement direction are constructed to provide a desired amount and type of holding strength on the tissue. In an embodiment, the engagement angle is at least 90 degrees, as shown in FIG. 5A. In another embodiment, the penetrator takes an engagement angle of approximately 180 degrees such that the penetrator retroflexes to lie in a plane that is parallel to the plane of the longitudinal axis of the shaft 104 of the medical instrument, as shown in FIG. 5B. In yet another embodiment, the penetrator 120 passes through an engagement angle of about 180 degrees or more and re-emerges through the top surface of the tissue T, as shown in FIG. 5C. Preferably, the penetrator 120 forms an engagement angle sufficient to form a relatively closed loop between the penetrator 120 and the end effector 102 to provide sufficient strength for the medical instrument to manipulate tissue.

The prior art “hook”-type tissue graspers are typically formed of shape memory wire (e.g., Nitinol) that is shape set in the form of a hook. A limitation of these types of devices is the need for optimization of the “hook” portion of the device. The hook must be flexible enough to be retractable within the shaft of the device, but strong enough to hold tissue once extended. In addition, there is a limitation on the column strength of the exposed penetrator because it would be configured from the flexible shape memory material.

Accordingly, in several embodiments of the medical instruments described herein, the penetrator 120 is formed of a rigid material thereby providing sufficient column strength for penetration of tissue, cartilage, or soft bone. In several of these embodiments, the medical instrument includes an activation mechanism that is coupled to the control member 106 and that is adapted to move the penetrator 120 through its designed engagement angle.

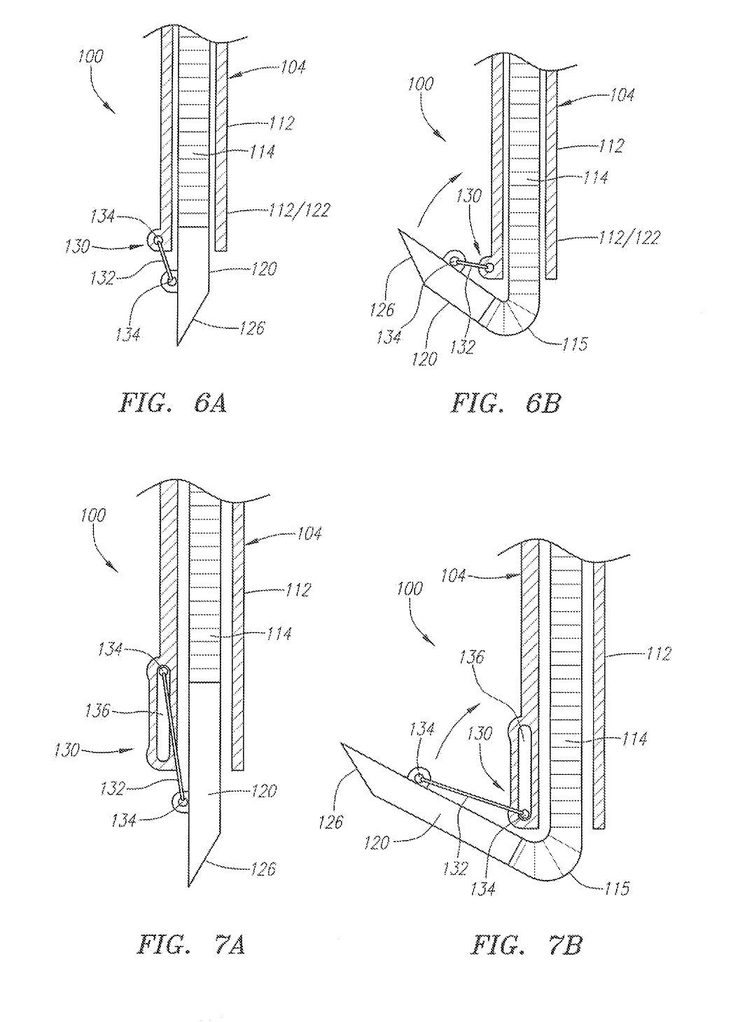

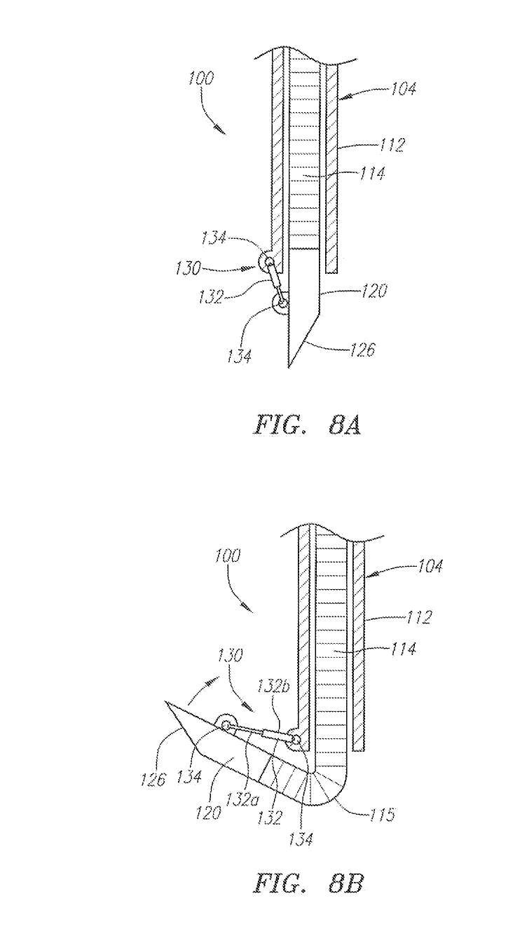

An example of a medical instrument 100 having an activation mechanism 130 suitable for moving the penetrator 120 through an engagement angle is shown in FIGS. 6A-B. The activation mechanism 130 includes a link 132 that is pivotably attached at a first end to the penetrator 120, and pivotably attached at a second end to the distal end of the sleeve 112 or the stop member 122. In an embodiment, the pivoting connectors 134 connecting the link 132 to the sleeve 112 and to the penetrator 120 are pins contained in a hole or slot formed on the respective member. In other embodiments, the connectors 134 comprise hinges or other rotating members. In an embodiment, the link 132 comprises a fixed length rigid wire, rod, or other suitable member.

In the embodiment shown in FIGS. 6A-B, a coiled-wire type pusher 114 is housed within the sleeve 112 and is connected at its distal end to the penetrator 120. The coiled wire pusher 114 is sufficiently flexible to be bent by the activation mechanism 130. The penetrator 120 is rotated by the activation mechanism 130 through an engagement angle of greater than 90 degrees as the pusher 114 is advanced distally within the sleeve 112, as shown in FIG. 6B.

The size and shape of the engagement angle is controlled by the lengths of the penetrator 120 and of the link 132. In another embodiment, the pusher 114 is adapted to form an elbow upon distal advancement, thereby increasing the amount of force applied by the penetrator 120 and/or enhancing the shape and stability of the end effector 102. For example, in an embodiment, the pusher 114 includes a bending portion 115 (see FIG. 6B) that includes a tube having transverse slots that facilitate bending of the tube while maintaining column strength and stability in the non-curling planes.

FIGS. 7A-B illustrate another embodiment of a medical instrument 100 that includes an activation mechanism 130 having a link 132 pivotably connected to the penetrator 120 by a connector 134. The link 132 is connected at its other end to the distal portion of the shaft 104 by a connector 134 that slides longitudinally within a slot 136 formed on the distal portion of the shaft 104. In this manner, the penetrator 120 is configured to move distally through a longer length of travel for a given length of the link 132, due to the travel of the connector 134 within the slot 136.

FIGS. 8A-B illustrate yet another embodiment of a medical instrument 100 that includes an activation mechanism 130 having a link 132 that is attached via a connector 134 at a first end to the penetrator 120 and by another connector at the other end to the distal portion of the shaft 104. In the embodiment shown, the link 132 is a telescoping member having an inner shaft 132a and outer tube 132b that provides the link 132 with the capacity to have a variable length. Accordingly, as the penetrator 120 is advanced distally, it causes the link 132 to lengthen, thereby providing an increased penetration depth of the penetrator 120.

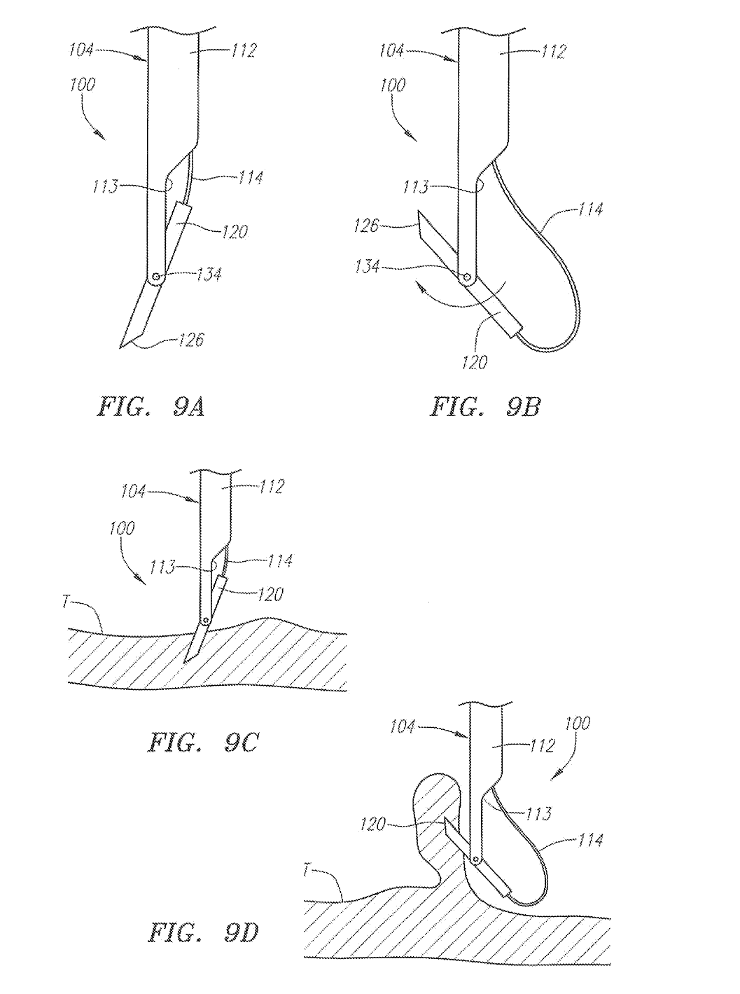

Turning to FIGS. 9A-D, another embodiment of a medical instrument 100 is shown. In the embodiment, the penetrator 120 is attached by a pivot 138 to the distal end of the shaft 104. The pivot 138 provides the penetrator 120 with the capacity for rotating around the axis of the pivot 138, thereby providing the ability for the penetrator to rotate through an engagement angle. A recess 113 is formed on the distal end of the shaft 104 to allow the penetrator 120 to rotate. In this embodiment, the penetrator 120 is moved through an engagement angle but does not move distally relative to the sleeve 112. In the embodiment shown, the pusher 114 comprises a drive wire that is sufficiently flexible that it is capable of bending out of its longitudinal plane as it is extended, as shown, for example, in FIG. 9B. This bending movement of the pusher 114 facilitates the rotation of the penetrator 120 about its fixed rotation axis and through its engagement angle.

The operation of the medical instrument is illustrated in FIGS. 9C-D. As shown in FIG. 9C, the medical instrument 100 is advanced first to bring the penetrator 120 to a position adjacent to the tissue T, and then advanced further to cause the penetrator 120 to penetrate the tissue T at a location of interest. After penetration, the pusher 114 is advanced to cause the penetrator 120 to rotate around the axis of the pivot 138, thereby engaging a portion of tissue T and providing the medical instrument 100 with the ability to push, pull, or otherwise manipulate the acquired tissue.

In several of the embodiments, the pusher 114 is advanced under control of the controller, such as the handle 106, as described above in relation to FIGS. 1A and 1D. The controller is actuated by the user to advance the pusher 114, thereby activating the penetrator 120. The activation speed is controlled by the user operating the controller (e.g., the handle 106), so as to move the penetrator 120 through its engagement angle at a desired speed. In an embodiment, the activation speed is sufficiently fast to cause the penetrator 120 to pass through the engagement angle and engage the target tissue in a fraction of a second, such as 0.001 seconds to 0.5 seconds. An appropriate speed of the penetrator 120 for the type of target tissue T causes the penetrator 120 to more effectively engage and grasp the tissue. In another embodiment, the activation speed is less fast, causing the penetrator 120 to pass through the engagement angle and engage tissue in a time of greater than 0.5 seconds.

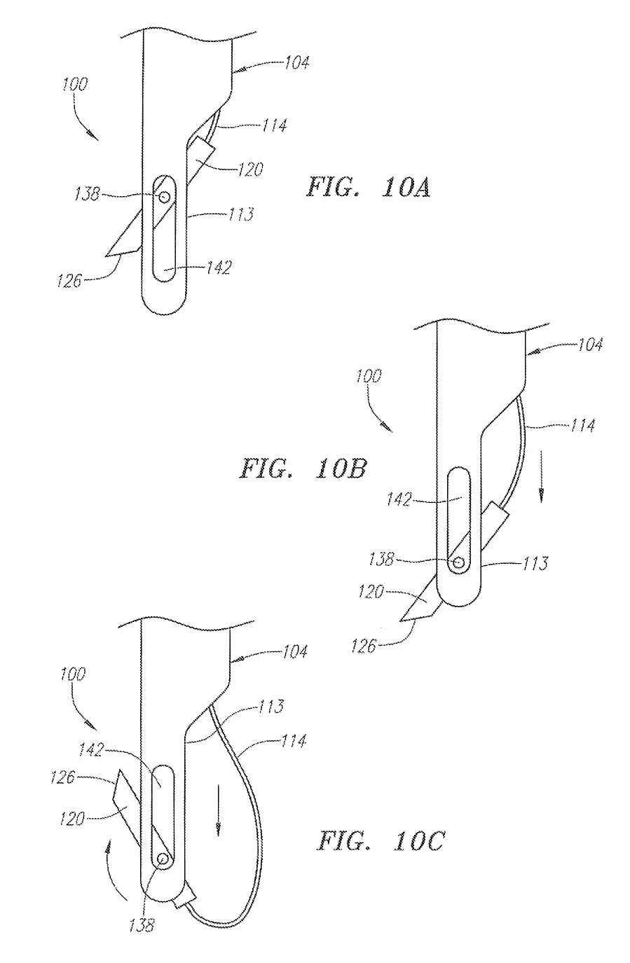

In FIGS. 10A-C, another embodiment of a medical instrument 100 is shown. The medical instrument 100 is otherwise similar to the embodiment described above in relation to FIGS. 9A-D, but is provided with a longitudinal slot 142 formed on the distal end of the shaft 104. The penetrator pivot 138 is configured to slide longitudinally within the slot 142, thereby providing the ability to move the penetrator 120 longitudinally relative to the shaft 104. As a result, the user is able to retract the penetrator 120 into the shaft 104 prior to activation, thereby providing an atraumatic state for the distal end of the medical instrument 100. The atraumatic state is useful in situations in which the device is being loaded through accessory devices or through the anatomy. The atraumatic state is also useful to allow the user to effectively cover and/or protect the penetrator 120 when the end effector 102 is in a confined or sensitive space prior to deployment of the penetrator 120.

Operation of the device is illustrated in FIGS. 10A-C. In FIG. 10A, the penetrator 120 is substantially retained within the distal end of the shaft 104 and is thereby retained in a substantially atraumatic state. Although the penetrator 120 is shown in FIG. 10A slightly rotated about the axis of the pivot 138, the penetrator 120 is also capable of being positioned fully within the shaft 104 and aligned fully longitudinally in its atraumatic state. In FIG. 10B, the pusher 114 has been advanced distally to cause the penetrator 120 to advance within the longitudinal slot 142 and to begin rotating through its engagement angle. In FIG. 10C, the pusher 114 has been fully advanced so as to cause the penetrator 120 to rotate through the full extent of the engagement angle.

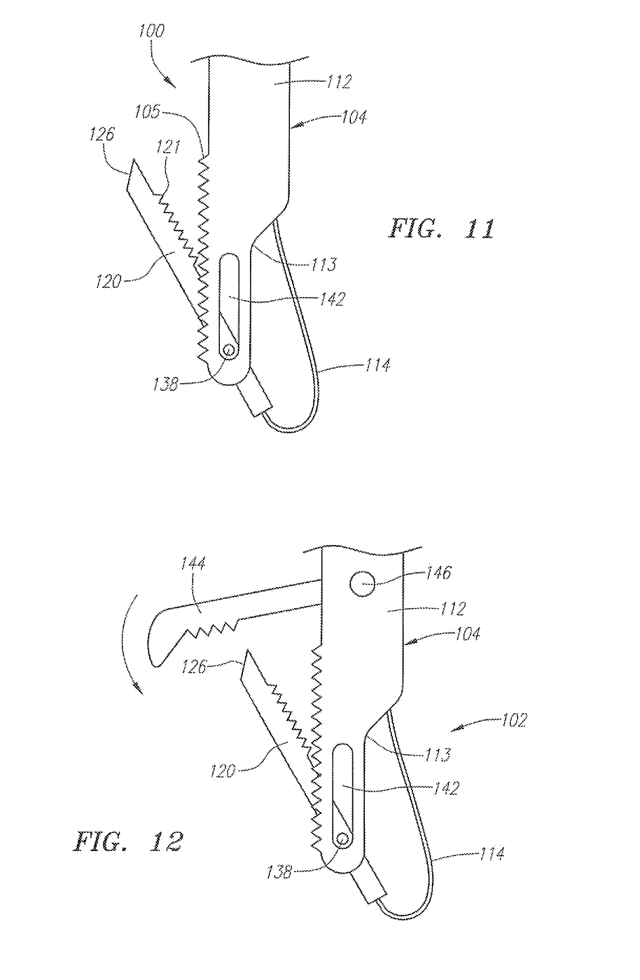

FIG. 11 illustrates another embodiment of the medical instrument. The medical instrument 100 is generally similar to the embodiment described above in relation to FIGS. 10A-B, but is provided with tissue-engaging surface features on the upper surface of the penetrator 120 and the outer surface of the distal end of the shaft 104. For example, in the embodiment shown in FIG. 11, the upper surface of the penetrator 120 includes a plurality of ridges 121 that are adapted to enhance the tissue engaging strength of the penetrator as it is activated. In addition, a plurality of ridges 105 are formed on the outer surface of the distal end of the shaft 104. The ridges 105 are adapted to enhance the tissue engaging strength of the shaft 104 to effectively grasp tissue that is trapped between the penetrator 120 and the shaft 104 after activation of the penetrator 120. In other embodiments, the tissue-engaging surface features comprise teeth, knurled surfaces, roughened surfaces, notches, other suitable surface irregularities, or any combination of the same. The penetrator 120 and outer surface of the distal end of the shaft 104 are thereby provided with the capacity to operate as a reverse grasper to grasp and retain tissue between their facing surfaces.

Turning next to FIG. 12, another embodiment of a medical instrument 100 is shown. The medical instrument is constructed similarly to the device described above in relation to FIG. 11, but includes a proximal jaw 144 that is attached to the shaft 104 by a hinge 146. The proximal jaw 144 is attached to the shaft 104 at a point proximal of the end effector 102, and is oriented such that the jaw closes downward and distally of the hinge 146, as shown in the Figure. In an embodiment, the proximal jaw 144 is positioned such that the jaw is able to engage and retain the tissue T that is retained between the penetrator 120 and the distal end of the shaft 104.

In several embodiments, the medical instruments 100 described herein are configured to work through existing endoscopes as an accessory. Accordingly, in some embodiments, the devices has a transverse dimension of no larger than 3 mm to fit the majority of conventional endoscope tool channels having working lumens. The medical instrument is also provided with a flexible shaft, and the end effector is preferably flexible and has a minimal rigid length to facilitate loading and removal from the scope.

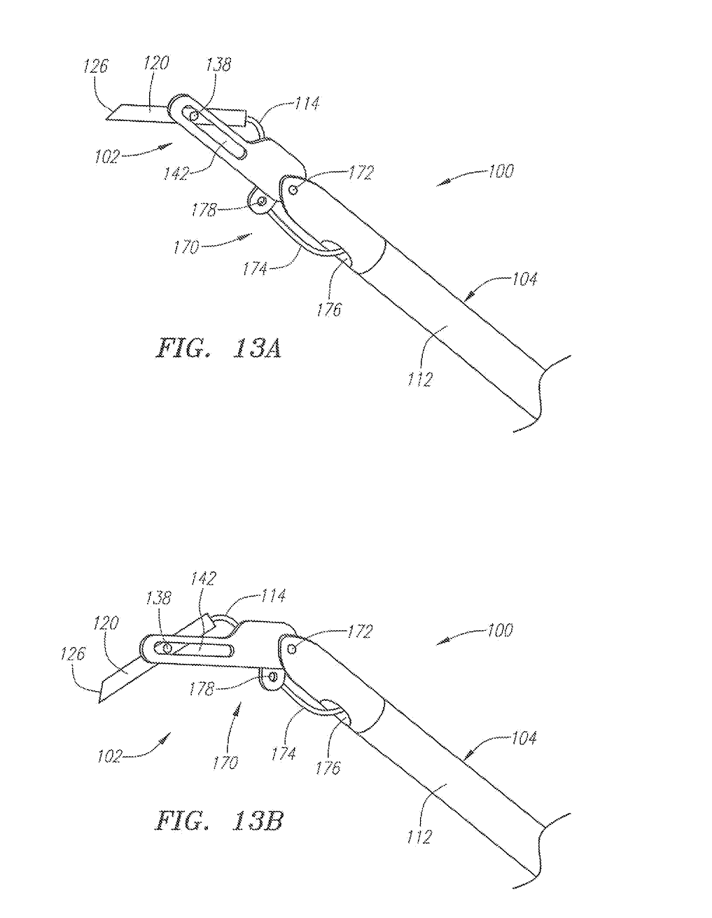

Although several embodiments of the medical instruments 100 described are adapted for use with a steerable endoscope or other overtube, in some embodiments the medical instrument 100 includes an articulation capability. For example, in FIGS. 13A-B, a medical instrument 100 similar to the embodiment described above in relation to FIGS. 10A-C is shown having an articulation mechanism 170 that is adapted to rotate the end effector 102 of the device around a joint formed at the distal end of the shaft 104. In an embodiment, the rotation joint comprises a hinge 172 or other pivot mechanism that rotatably connects the end effector 102 to the distal end of the shaft 104. An activator, such as a pull wire 174, extends through an exit port 176 formed near the distal end of the shaft 104, and extends proximally to the controller at the proximal end of the shaft 104. The distal end of the pull wire 174 is attached to a pivoting connector 178 formed on the end effector 102. Accordingly, as the pull wire 174 is retracted (proximally) and advanced (distally), the end effector 102 is caused to articulate through a range of motion relative to the longitudinal axis of the shaft 104. See FIGS. 13A-B. This articulation provides the user with the ability to move the end effector 102 to a position suitable for engaging and manipulating the tissue T.

The medical instruments described herein are adapted for use in engaging, penetrating, grasping, and manipulating tissue during open surgery, laparoscopic surgery, endoscopic surgery, or translumenal surgery. In particular, the medical instruments are adapted to engage the soft, multilayer tissue of a human or animal stomach in an endolumenal approach. Alternatively, the medical instruments may be used to engage other human or animal gastric tissue, peritoneal organs, external body surfaces, or tissue of the lung, heart, kidney, bladder, or other body tissue. The instruments are particularly useful for engaging, penetrating, grasping, and manipulating tissue that is difficult to engage using conventional graspers, which frequently occurs during translumenal surgical procedures (e.g., natural orifice translumenal endoscopic surgery, or “NOTES”). Several translumenal procedures are described in U.S. patent application Ser. No. 10/841,233, Ser. No. 10/898,683, Ser. No. 11/238,279, Ser. No. 11/102,571, Ser. No. 11/342,288, and Ser. No. 11/270,195, which are hereby incorporated by reference. The medical instruments described herein are suitable for use in combination with, for example, the endoluminal tool deployment systems described in U.S. patent application Ser. No. 10/797,485, which is hereby incorporated by reference. In particular, the tool deployment systems described in the '485 application includes one or more lumens suitable for facilitating deployment of the medical instruments described herein to perform or assist in performing endoscopic, laparoscopic, or NOTES diagnostic or therapeutic procedures. In addition, the medical instruments described herein are suitable for use in combination with, or instead of, the methods and instruments described in U.S. patent application Ser. No. 11/412,261, which is also incorporated by reference herein.

Although various illustrative embodiments are described above, it will be evident to one skilled in the art that various changes and modifications are within the scope of the invention. It is intended in the appended claims to cover all such changes and modifications that fall within the true spirit and scope of the invention.

What is claimed is:

1. Apparatus for penetrating and engaging tissue comprising:
a control member;
a tissue penetrating member;
an activation mechanism responsive to said control member and operatively coupled to said tissue penetrating member; and
an elongated flexible member extending between and coupled to each of said control member and said tissue penetrating member;
wherein said tissue penetrating member comprises a rigid member pivotably attached to said elongated flexible member.

2. The apparatus of claim 1, wherein said control member comprises a handle.

3. The apparatus of claim 2, wherein said handle comprises a ratcheting mechanism.

4. The apparatus of claim 2, wherein said handle comprises a pusher block slidably received within a main body, and an actuation arm connected to said pusher block by a linkage.

5. The apparatus of claim 1, wherein said elongated flexible member comprises a shaft having a sleeve and a pusher.

6. The apparatus of claim 1, wherein said activation mechanism comprises a drive wire attached to said penetrating member and adapted to move said penetrating member around its pivotable attachment to said elongated flexible member.

7. The apparatus of claim 1, wherein said penetrating member comprises a beveled needle.

8. The apparatus of claim 1, further comprising a frame defining an elongated slot formed on said elongated flexible member, and wherein said penetrating member is capable of translation motion restricted by said frame.

9. The apparatus of claim 1, wherein said activation mechanism comprises a drive wire.

10. The apparatus of claim 8, wherein said drive wire is flexible.

11. The apparatus of claim 1, wherein said elongated flexible member comprises a composite shaft.

12. The apparatus of claim 11, wherein said composite shaft comprises polyetheretherketone.

13. A method for penetrating and grasping tissue, comprising:
providing an instrument having a tissue penetrating member at a location adjacent to a tissue site;
causing said tissue penetrating member to penetrate tissue;
rotating said tissue penetrating member; and
manipulating said instrument to move said tissue from its natural position.

14. The method of claim 13, wherein the tissue site is a hollow body organ.

15. The method of claim 13, wherein the tissue penetrating member is provided at the location endoluminally.

16. The method of claim 13, wherein the tissue penetrating member is provided at the location laparoscopically.

17. The method of claim 13, wherein the tissue penetrating member is provided at the location by advancing the instrument through a natural body orifice.

18. The method of claim 13, wherein causing said tissue penetrating member to penetrate tissue includes moving said tissue penetrating member through a slot formed on said instrument.
